(19) United States
(12) Patent Application Publication
(10) Pub. No.: US 2009/0147077 A1
(43) Pub. Date: Jun. 11, 2009

(54) LIGHT-SOURCE CONTROL SYSTEM, SHUTTER CONTROL SYSTEM, ENDOSCOPE PROCESSOR, AND ENDOSCOPE SYSTEM

(75) Inventors: Nobuhiro TANI, Tokyo (JP); Noriko IRIYAMA, Saitama (JP)

Correspondence Address: GREENBLUM & BERNSTEIN, P.L.C., 1950 ROLAND CLARKE PLACE, RESTON, VA, 20191, US

(73) Assignee: HOYA CORPORATION, Tokyo (JP)

(21) Appl. No.: 12/327,892

(22) Filed: Dec. 4, 2008

(30) Foreign Application Priority Data
Dec. 5, 2007 (JP) 2007-314996

(51) Int. Cl. A61B 1/04

(52) U.S. Cl. 348/68

(57) ABSTRACT

A light-source control system including a detector and a controller is provided. The detector detects an output period. The XY-address type imaging device generates an image signal comprising a plurality of pixel signals. The pixel signals vary according to the signal charges. The pixels generate signal charges according to the amount of light received during a storing period. The storing period comprises a common-period and a variable-period. The common-period is simultaneous for all of pixel-rows. The variable-period varies according to each of the pixel-rows that are arranged in a first direction. Series of the pixel signals corresponding to the pixels that are arranged in the same pixel-row are output in order of the pixel-rows during the output period. The controller controls a light-source to suspend the emission of illumination light during the output period.

### BACKGROUND OF THE INVENTION

1. Field of the Invention

The present invention relates to the control of a light-source or a shutter with the goal of lowering the distortion of a moving image of a moving object captured by an XY-address type imaging device, such as a CMOS imaging device, which captures an optical image by line exposure.

2. Description of the Related Art

An electronic endoscope having an imaging device at a head end of an insertion tube is known as an apparatus for photographing and/or filming a moving object. CCD imaging devices have typically been used in prior electronic endoscopes. On the other hand, Japanese Unexamined Patent Publication No. 2002-58642 proposes that a CMOS imaging device is used for an electronic endoscope in order to reduce power consumption and manufacturing cost.

However, because a CMOS imaging device generally captures an optical image by line exposure, there is a troublesome distortion in the image of quick moving object captured by a CMOS imaging device.

### SUMMARY OF THE INVENTION

Therefore, an object of the present invention is to provide a light-source control system and shutter control system which reduces the distortion appearing in an image of a moving object captured by an XY-address type imaging device, such as CMOS imaging device, which captures an optical image by line exposure.

According to the present invention, a light-source control system comprising a detector and a controller is provided. The detector detects an output period if an XY-address type imaging device is drive to generate an image signal. The XY-address type imaging device comprises a plurality of pixels which are arranged in first and second directions. The image signal comprises a plurality of pixel signals corresponding to the pixels. The pixel signals vary according to signal charges. The pixels generate the signal charges according to the amount of light received during a storing period. The storing period comprises a common-period and a variable-period. The common-period is simultaneous for all of pixel-rows. The variable-period varies according to each of the pixel-rows. The pixel-rows comprise the pixels that are arranged in said first direction. Series of the pixel signals corresponding to the pixels that are arranged in the same pixel-row are output in order of the pixel-rows during the output period following the common-period. The controller controls a light-source to suspend the emission of illumination light during said output period. The illumination light is shone on an object of which image is captured by the XY-type imaging device.

According to the present invention, a shutter control system comprising a detector and a controller is provided. The detector detects an output period if an XY-address type imaging device is driven to generate an image signal. The XY-address type imaging device comprises a plurality of pixels which are arranged in first and second directions. The image signal comprises a plurality of pixel signals corresponding to the pixels. The pixel signals vary according to signal charges. The pixels generate the signal charges according to the amount of light received during a storing period. The storing period comprises a common-period and a variable-period. The common-period is simultaneous for all of pixel-rows. The variable-period varies according to each of the pixel-rows. The pixel-rows comprise the pixels that are arranged in said first direction. Series of the pixel signals corresponding to the pixels that are arranged in the same pixel-row are output in order of the pixel-rows during the output period following the common-period. The controller controls a shutter to block light toward the XY-address type imaging device during the output period. The shutter is mounted on a light-receiving surface of the XY-address type imaging device.

### BRIEF DESCRIPTION OF THE DRAWINGS

The objects and advantages of the present invention will be better understood from the following description, with reference to the accompanying drawings in which:

FIG. 1 is a block diagram showing the internal structure of an endoscope system having a light-source control system of a first embodiment of the present invention;

FIG. 2 is a block diagram showing the internal structure of a light-source unit;

FIG. 3 is a block diagram showing the structure of an imaging device;

FIG. 4 is a circuit diagram showing the internal structure of pixel;

FIG. 5 is a timing chart illustrating the timing used to drive the imaging device focusing on the output operation of pixel signals which one field of an image signal consists of;

FIG. 6 is a timing chart illustrating the timing used to drive the imaging device and the light source focusing on the output operation of successive fields of image signals in the first embodiment;

FIG. 7 is a block diagram showing the internal structure of an endoscope system having a shutter control system in a second embodiment of the present invention; and

FIG. 8 is a timing chart illustrating the timing used to drive the imaging device and to switch the shutter focusing on the output operation of successive fields of image signals in the second embodiment.

### DESCRIPTION OF THE PREFERRED EMBODIMENTS

The present invention is described below with reference to the embodiments shown in the drawings.

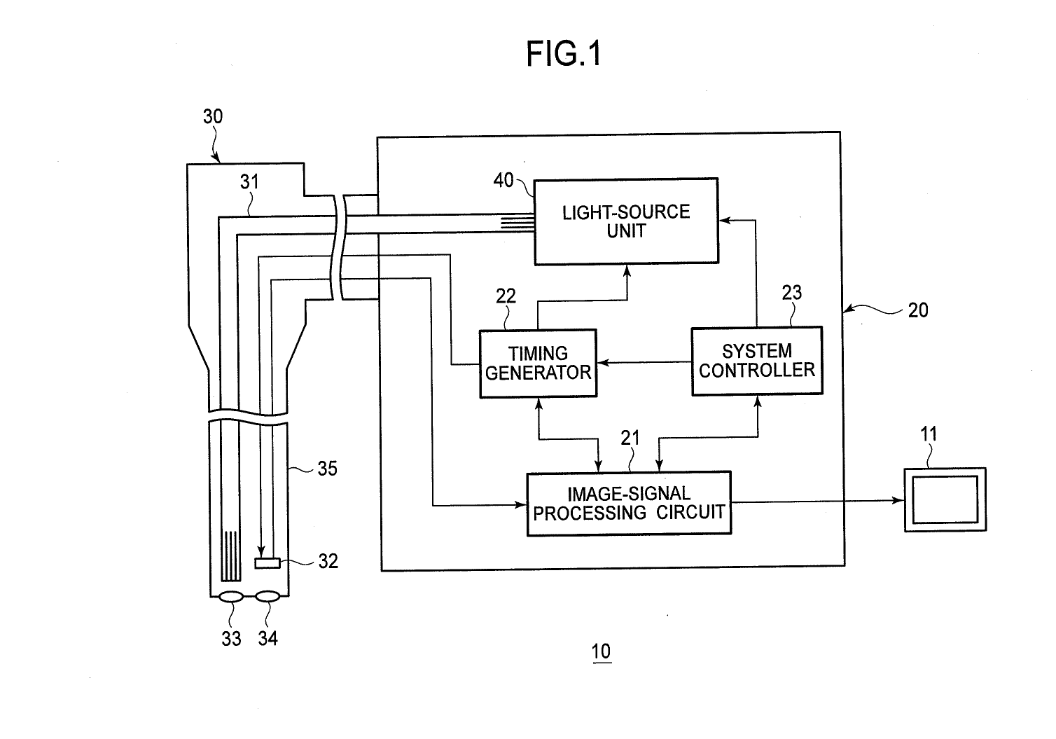

In FIG. 1, an endoscope system 10 comprises an endoscope processor 20, an electronic endoscope 30, and a monitor 11. The endoscope processor 20 is connected to the electronic endoscope 30 and the monitor 11.

The endoscope processor 20 emits illumination light to illuminate a required object. The illuminated object is photographed and/or filmed by the electronic endoscope 30, and then the electronic endoscope 30 generates an image signal. The image signal is sent to the endoscope processor 20.

The endoscope processor 20 carries out predetermined signal processing on the received image signal. The image signal, having undergone predetermined signal processing is sent to the monitor 11, where an image corresponding to the received image signal is displayed.

The endoscope processor 20 comprises a light-source unit 40, an image-signal processing circuit 21, a timing generator 22, a system controller 23 (a detector), and other components. As described below, the light-source unit 40 emits illumination light for illuminating a desired object toward the incident end of light guide 31. In addition, as described below, the image-signal processing circuit 21 carries out predetermined signal processing on the image signal. In addition, the timing generator 22 times some operations of the components of the endoscope system 10. In addition, the system controller 23 controls the operations of all components of the endoscope system 10.

By connecting the endoscope processor 20 to the electronic endoscope 30, the light-source unit 40 and a light-guide 31 mounted in the electronic endoscope 30 become optically connected. In addition, by connecting the endoscope processor 20 to the electronic endoscope 30, electrical connections are made between the image-signal processing circuit 21 and the imaging device 32 mounted in the electronic endoscope 30, and between the timing generator 22 and the imaging device 32.

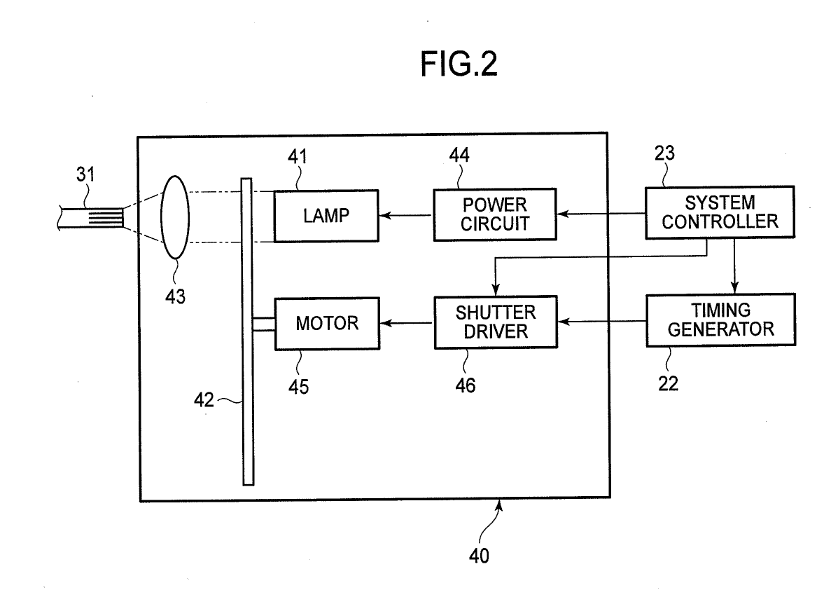

As shown in FIG. 2, the light-source unit 40 comprises a lamp 41, a rotary shutter 42, a condenser lens 43, a power circuit 44, a motor 45, a shutter driver 46 (controller), and other components.

The lamp 41 is, for example a xenon lamp or halogen lamp, and emits white light. The rotary shutter 42 and the condenser lens 43 are mounted on an optical path of white light from the lamp 41 to the incident end of the light guide 31.

The rotary shutter 42 has a circular plate shape and has an aperture area and a blocking area. When white light should be emitted from the light source unit 40, the aperture area is inserted into the optical path of white light. On the other hand, when the emission of white light should be suspended, the blocking area is inserted into the optical path of white light, blocking white light. The rotary shutter 42 is driven by the motor 45. The switching cycle between the emission of and the suspension of the emission of white light is adjusted by controlling the rotation speed of the motor 45.

The shutter driver 46 controls the motor 45 so that the motor 45 drives the rotary shutter 42. The shutter driver 46 controls the motor on the basis of a clock signal and an output-period detection signal transmitted from the timing generator 22 and the system controller 23, respectively, as described in detail later.

White light emitted by the lamp 41 is condensed by the condenser lens 43, and is directed to the incident end of the light guide 31. The power circuit 44 supplies the lamp 41 with power. The system controller 23 switches power supply to the lamp 41 from the power circuit 44 to power the lamp 41 on and off.

Next, the structure of the electronic endoscope 30 is explained in detail. As shown in FIG. 1, the electronic endoscope 30 comprises the light guide 31, the imaging device 32, a diffuser lens 33, an object lens 34, and other components.

The incident end of the light guide 31 is mounted in a connector (not depicted) which connects the electronic endoscope 30 to the endoscope processor 20. And the other end, hereinafter referred to as the exit end, is mounted at the head end of an insertion tube 35 of the electronic endoscope 30. As described above, white light emitted by the light-source unit 40 arrives at the incident end of the light guide 31. The light is then transmitted to the exit end. The light transmitted to the exit end illuminates a peripheral area near the head end of the insertion tube 35 through the diffuser lens 33.

An optical image of reflection light of the object illuminated by white light reaches a light-receiving surface of the imaging device 32 through the object lens 34. The clock signal and the field signal are transmitted from the timing generator 22 to the imaging device 32. The imaging device 32 generates an image signal corresponding to the optical image reaching the light-receiving surface on the basis of the clock signal and the field signal.

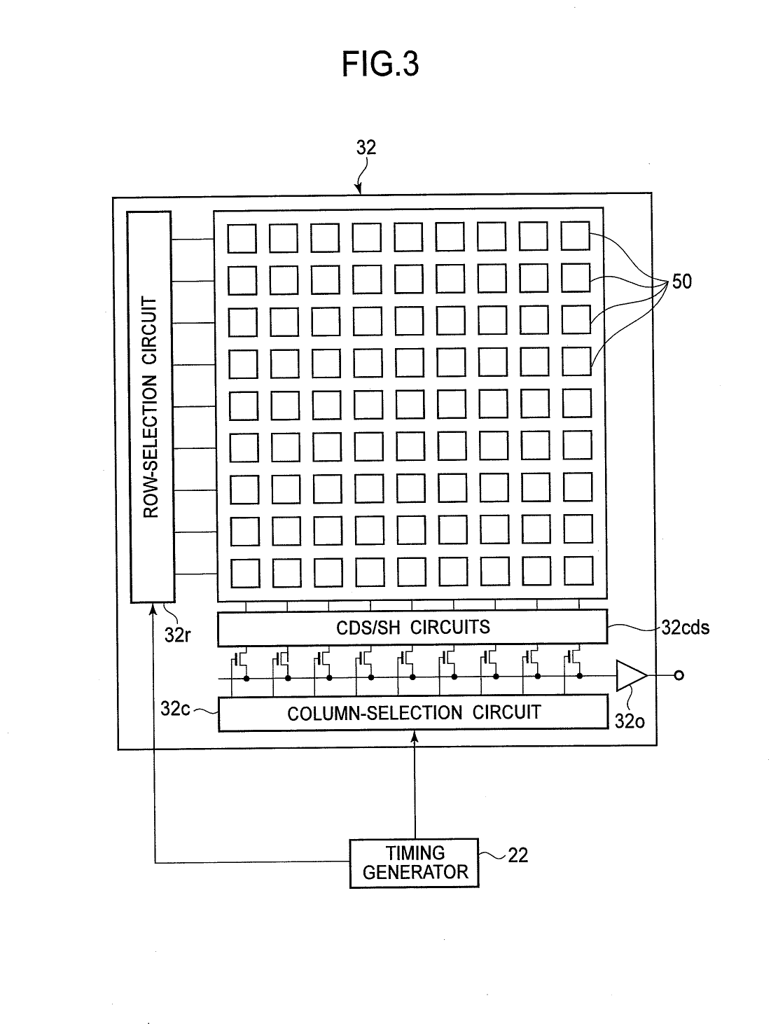

The imaging device 32 is a CMOS imaging device, which is one of an XY-address type imaging device. As shown in FIG. 3, a plurality of pixels 50 are arranged in a grid on the light-receiving surface of the imaging device 32. Each pixel 50 generates a pixel signal according to the amount of light received by the pixel 50. The pixel signals are output one by one in order via the output block 32o. The image signal is comprised of pixel signals output during a single field period, which is half of one cycle of the field signal. A pixel 50 that should be ordered to output the pixel signal is selected by a row-selection circuit 32r and a column-selection circuit 32c.

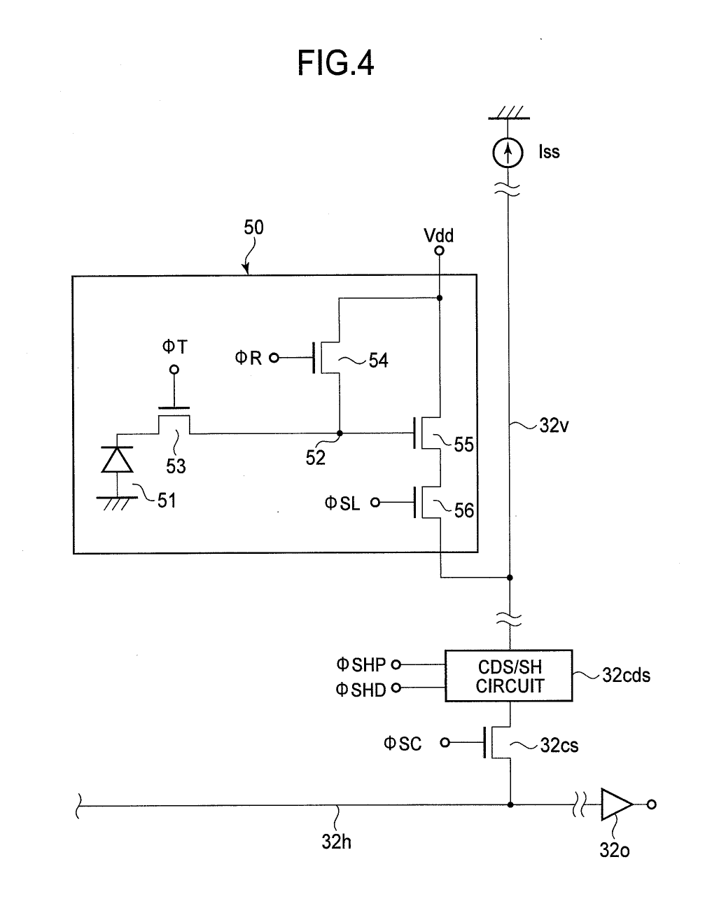

The internal structure of each pixel 50 is explained below using FIG. 4. The pixel 50 comprises a photodiode (PD) 51, a floating diffusion (FD) 52, a transfer transistor 53, a reset transistor 54, an amplification transistor 55, and a row-selection transistor 56.

A signal charge is generated according to the amount of light received and stored by photoelectric conversion of the PD 51. The stored signal charge is transmitted to the FD 52 when the transfer transistor 53 is switched on. The FD 52 is a capacitor, of which electrical potential varies according to the stored signal charge.

When the reset transistor 54 is switched on, the FD 52 is reset. Then, the signal charge stored in the FD 52 is swept to a power source, hereinafter referred to as Vdd. Then, the electrical potential of the FD 52 is reset to an electrical potential of the Vdd.

The amplifier transistor 55 outputs a voltage signal according to the electrical potential of the FD 52 to the row-selection transistor 56, by adjusting output impedance.

A vertical output line 32v is mounted along every column of pixels 50. The vertical output line 32v is connected to all pixels arranged in the same column. When the row-selection transistor 56 is switched on, the voltage signal is output to the vertical output line 32v. By separately switching on each of the row-selection transistors 56, voltage signals can be separately output from pixels 50 connected to the same vertical output line 32v.

The vertical output lines 32v are separately connected to CDS/SH circuits 32cds. An electrical potential of the FD 52 includes reset noise after resetting the FD 52. A voltage signal corresponding to a signal charge received after resetting includes the reset noise. The CDS/SH circuit 32cds removes the reset noise included in the voltage signal by correlated double sampling, and then a voltage signal according to the signal charge stored by the PD 51 is output as a pixel signal.

The CDS/SH circuits 32cds are connected to a horizontal output line 32h via column-selection transistors 32cs. By switching on the column-selection transistors 32cs one by one in order, pixel signals generated by the CDS/SH circuits 32cds in all columns can be separately output to the image-signal processing circuit 21 via the horizontal output line 32h and the output block 32o.

A transfer signal line (not depicted) is mounted along each row of pixels 50. The transfer signal line is connected to transfer transistors 53 in all the pixels arranged in a given row. A transfer signal, hereinafter referred to as φT, is sent to all transfer signal lines. The φT has high and low states. The φTs, which are sent to each row of the transfer signal line, is set to the high state at different times for each row. When the φT is set to the high state, the transfer transistor 53 is switched on, and consequently the transfer transistor 53 becomes conductive.

A reset signal line (not depicted) is mounted along each row of pixels 50. The reset signal line is connected to reset transistors 54 in all the pixels arranged in a given row. A reset signal, hereinafter referred to as φR, is sent to all reset signal lines. The φR has high and low states. The φRs, which are sent to each of row of the reset signal line, is set to the high state at different times for each row. When the φR is set to the high state, the reset transistor 54 is switched on, making the reset transistor 54 conductive.

A row-selection signal line (not depicted) is mounted along each row of pixels 50. The row transfer signal line is connected to row-selection transistor 56 in all the pixels arranged in a given row. A row selection signal, hereinafter referred to as φSL, is sent to all row-selection signal lines. The φSL has high and low states. The φSLs, which are sent to each row of the row-selection signal line, is set to the high state at different times for each row. When the φSL is set to the high state, the row-selection transistor 56 is switched on, making the row-selection transistor 56 conductive.

Column selection signals, hereinafter referred to as φSC, are transmitted separately to the column-selection transistors 32cs. While the φSC is set to the high state, the column-selection transistor 32cs is switched on, making the column-selection transistor 32cs conductive.

The row-selection circuit 32r outputs the φT, φR, and φSL to the transfer signal line, the reset signal line, and the row-selection signal line to control the switching operations of the reset transistor 54 and the row-selection transistor 56. In addition, the row-selection circuit 32r controls the correlated double sampling operation of the CDS/SH circuits 32cds. The column-selection circuit 32c outputs the φSCs to the column-selection transistors 32cs to control the switching operation of the column-selection transistor 32cs.

The row-selection circuits and the column-selection circuit 32r and 32c control the switching operations and the correlated double sampling operation on the basis of the clock signal and the field signal transmitted from the timing generator 22.

A series of pixel signals output during a field period is transmitted as a pixel signal to the image-signal processing circuit 21. The image-signal processing circuit 21 carries out predetermined signal processing on the received image signal.

In addition, φSLs for pixels 50 in the first and the mth row for which pixel signals are output first and last during each field period, respectively, hereinafter referred to as φSL1 and φSLm, are transmitted to the system controller 23 via the image signal processing unit 21. The system controller 23 sends the output-period detection signal to the shutter driver 46 since the φSL1 is switched into high state until the φSLm is switched into low state from the high state.

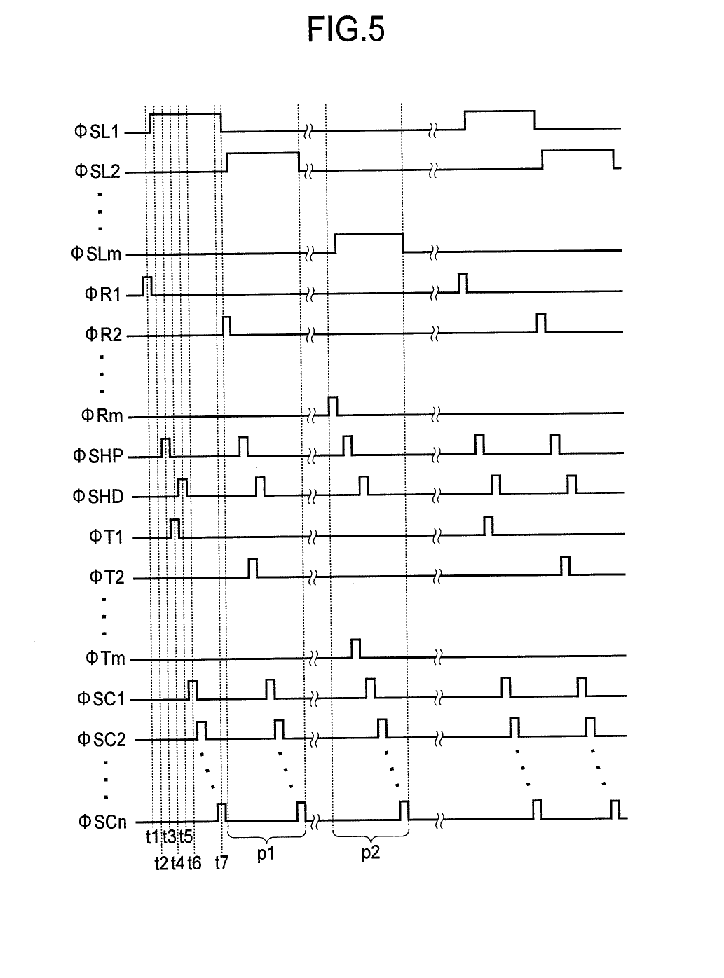

The operation of the imaging device 32 for outputting one field of an image signal is described below with reference to FIG. 5.

At time t1, the φR for pixels 50 in the first row, hereinafter referred to as φR1, is set to the high state, and then, the reset transistors 54 in the pixels 50 arranged in the first row is switched on. By switching on the reset transistors 54, the FDs 52 get reset.

At time t2 soon after the FDs 52 are reset, the φSL1 is set to the high state, and then, the pixel signals can be output from the pixels 50 in the first row. The φSL1 is kept in the high state until the output of all pixel signals of the pixels 50 in the first row finishes, when the φSC for pixels 50 in the nth column, hereinafter referred to as φSCn, is set to the high state.

At time t3, a pre-hold signal, hereinafter referred to as φSHP, is set to the high state, and then, the electrical potential of the reset FDs 52 of all the pixels 50 in the first row are sampled and held by the CDS/SH circuits 32cds corresponding to each column.

At time t4, the φT for pixels 50 in the first row, hereinafter referred to as φT1, is set to the high state, and then, the signal charges stored by the PDs 51 in the pixels 50 in the first row is transferred to the FDs 52.

At time t5, a data hold signal, hereinafter referred to as φSHD, is set to the high state, and then, the electrical potential of the FDs 52 receiving the signal charges of all the pixels 50 in the first row are sampled and held by the CDS/SH circuits 32cds. The CDS/SH circuits 32cds have a subtractor circuit which generates a pixel signal by subtracting the electrical potential of the reset FDs 52s from the electrical potential of the FDs 52 receiving the signal charges. The generated pixel signal is then able to be output from the CDS/SH circuits 32cds.

At time t6, the φSC for the first column, hereinafter referred to as φSC1, is set to the high state. Then, the column-selection transistor 32cs of the first column becomes conductive, and the pixel signal held by the CDS/SH circuit 32cds in the first column is output to the image-signal processing circuit 21 via the horizontal output line 32h and the output block 32o.

Following the output of the pixel signals in the first column, the φSC for the second column, hereinafter referred to as φSC2, is set to the high state. Then, the pixel signal held by the CDS/SH circuit 32cds in the second column is output to the image-signal processing circuit 21 via the horizontal output line 32h and the output block 32o. Next, the φSC for each of all columns is set to the high state one by one, and the pixel signals in each column in the first row are output one by one.

At time t7, the φSCn is set to the high state. Then, the pixel signal is output from the CDS/SH circuit 32cds of the nth column, which is the last column, and the output of the pixel signals from all the pixels 50 in the first row completes. In addition, at the same time, the φSL1 is set to the low state.

Next to output the pixel signals in the first row, the φR for the pixels 50 in the second row, hereinafter referred to as φR2, is set to the high state, and then, pixel signals of the second row start to be output. The pixel signals are output from the pixels 50 in the second row as in the same operations carried out at times t1-t7 (see period p1).

From that point, the pixel signals of all the rows are output by setting the φT, φR, and φSL of each row into the high state as in the same operations carried out at the times t1-t6.

The pixel signals of the mth row, which is the last row, are output during the period p2 when the φSLm is kept at the high state. When all the pixel signals of the first to last row are output, the output of one field of an image signal is complete.

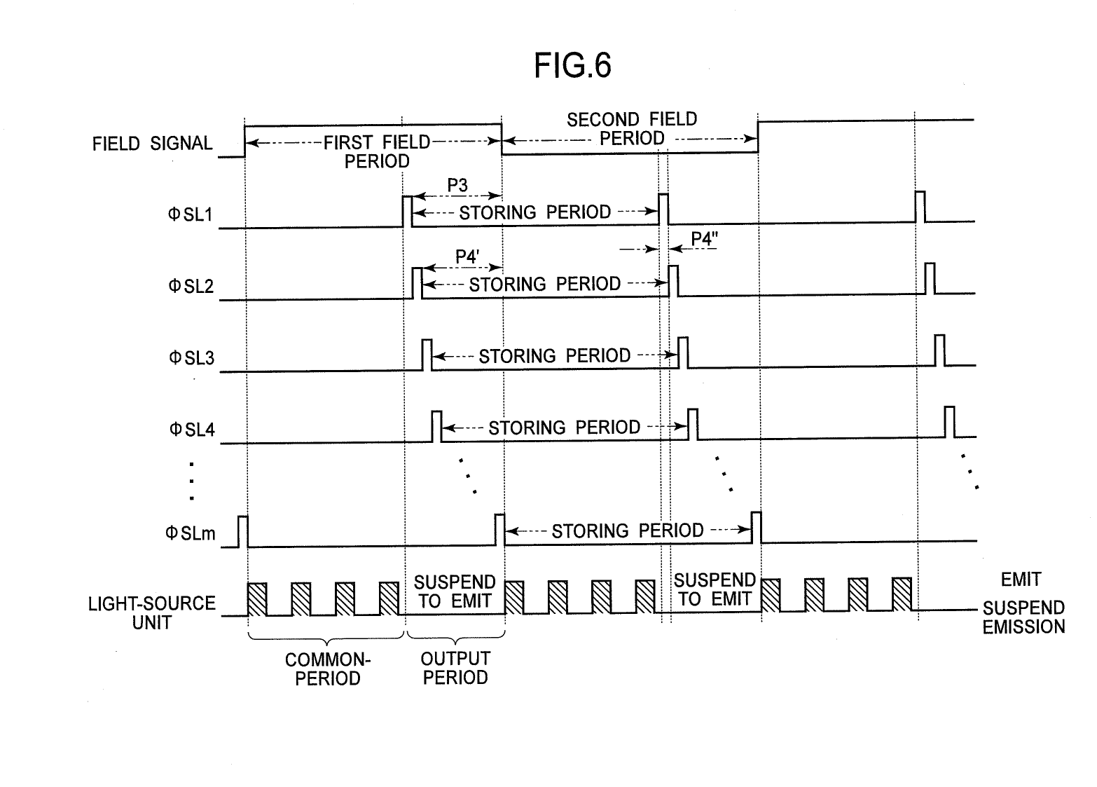

The operation of the imaging device 32 and the light-source unit 40 to output successive fields of image signals is described below with reference to FIG. 6.

The timing generator 22 generates a field signal with a cycle of 1/30 seconds, and sends it to the imaging device 32 and the light-source unit 40. As described above, a half of one cycle of the field signal, which is a period during either the high or low state of the field signal, is defined as the field period.

The field period is divided into a common-period and an output period (see the bottom of FIG. 6). A point at which the field signal is switched between high and low states is defined as the starting time of the common-period. The period from the completion of the common period to the time to next switching of the field signal between high and low states is defined as the output period.

During the output period, pixel signals of all rows are output one by one in order. The pixel signals of the first row are output during the period during when the φSL1 is kept at the high state shown in FIG. 6, just as in FIG. 5. The periods during which the φSL1 is kept at the high state in FIGS. 5 and 6 are equivalent. In addition, the pixel signals of the second—mth row are also output during the period during which the φSL2-φSLm is kept at the high state in FIG. 6, respectively.

As described above, signal charges stored by the PDs 51 are transferred to the FDs 52 by making the transfer transistors 53 conductive. When the conductive state of the transfer transistors 53 is suspended, the PDs 51 start to generate and store signal charges. Accordingly, the period during which the conductive state of the transfer transistors 53 of each row is kept suspended is a storing period during which signal charges of the corresponding row are kept generated and stored (see storing period). The times when signal charges are transferred to the FDs 52 differs between all the rows. Accordingly, the storing periods differs between all the rows. In FIG. 6, the time when the φSL is kept at the high state is regarded as the time when the φT is kept at the high state.

A period which is a part of the output period after completing the output of the pixel signals of the first row during the first field period is defined as a variable-period for the first row (see “p3”) The combination of the variable-period for the first row and the common-period following the variable-period for the first row is the storing period for the pixels 50 in the first row. Signal charges are generated and stored in all the pixels 50 in the first row according to the amount of light received during the storing period for the pixels 50 in the first row. The signal charges will be output as pixel signals of the first row of the second field period.

A period which is a part of the output period after completing to output the pixel signals of the second row during the first field period is defined as a first variable-period for the second row (see “p4′”). The period which is a part of the output period before starting to output the pixel signals of the second row during the second field period is defined as a second variable-period for the second row (see “p4″”). The combination of the first variable-period for the second row, the common-period following the first variable-period for the second row, and the second variable-period for the second row is the storing period for the pixels 50 in the second row. Signal charges are generated and stored in all the pixels 50 in the second row according to the amount of light received during the storing period for the pixels 50 in the second row. And the signal charges will be output as pixel signals of the second row of the second field period.

As in the first and second rows, the combination of a common-period and parts of output periods before and/or after the common period is defined as the storing period of each row. The parts of the output periods for a row differ from those for the other rows, and start and complete their output at a time different from those of the other rows.

The rotary shutter 42 is driven so that the light-source unit 40 emits a pulse of white light only during the common-period (see the column of “light-source unit”). In addition, the rotary shutter 42 is driven on the basis of the output-period detection signal transmitted by the system controller 23 so that the emission of the light-source unit 40 is suspended during the output period.

Accordingly, an optical image produced by the light reflected from an object is captured by all the pixels 50 in the imaging device 32 only during the common-period, which is the same for all rows during a given field period even though the storing periods differ by row. Consequently, signal charges are actually generated and stored by the pixels in all the rows during the same common-period.

In the above first embodiment, periods and times during which light based on the illumination light is actually received by all the pixels 50 can be made to accord under the condition that no light excepting for the illumination light is cast on an object, such as, the typical observation case using an electrical endoscope. Accordingly, if an optical image of a moving object should be captured by a CMOS imaging device, the distortion appearing in an image of a moving object will be reduced.

If an object illuminated by a pulse of white light is photographed and/or filmed without controlling the period when the illumination light is shone on the object, in contrast to the first embodiment above, the numbers of rows for white light emission pulse may differ. If the number of pulses used to illuminate each row varies, the cumulative amount of light cast on the rows will also vary. To solve this problem, in the first embodiment above, the number of pulses used to illuminate the rows are made to accord, and then, total amounts of light illuminated subject for all rows are made to accord also. Accordingly, uneven luminance for rows caused by the difference of total amounts of illuminated light for rows in a displayed image can be prevented.

Next, a shutter control system of the second embodiment is explained. The primary difference between the second embodiment and the first embodiment is the method of blocking light incident on the light-receiving surface of the imaging device during a period other than the common-periods. The second embodiment is explained mainly with reference to the structures that differ from those of the first embodiment. Here, the same index numbers are used for the structures that correspond to those of the first embodiment.

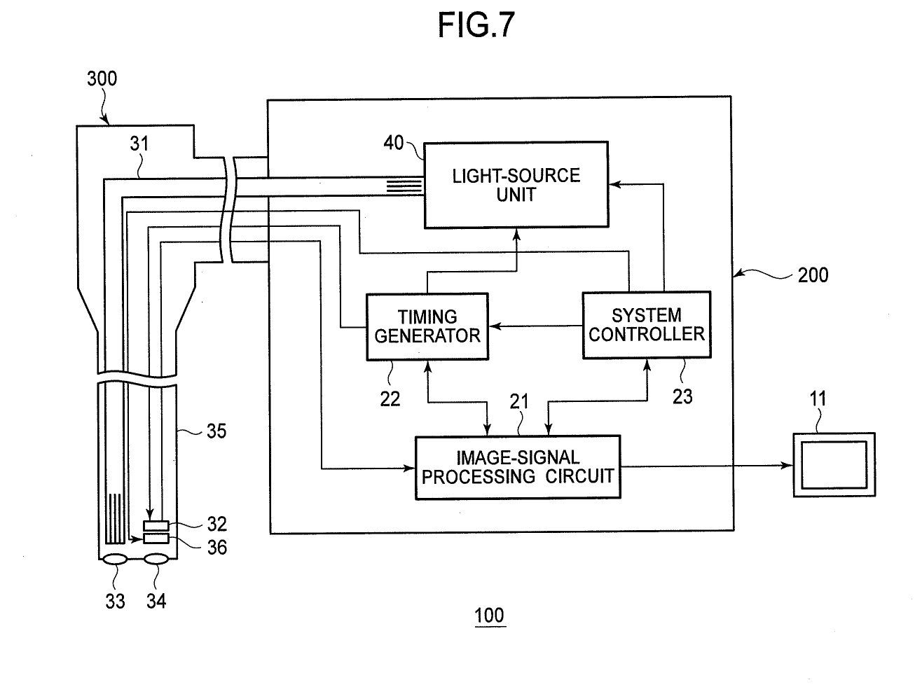

As shown in FIG. 7, an endoscope processor 200 comprises a light-source unit 40, an image-signal processing circuit 21, a timing generator 22, a system controller 23 (controller), and other components, as in the first embodiment.

The light-source unit 40 emits white light cast on an object toward the incident end of light guide 31, as in the first embodiment. In addition, the image-signal processing circuit 21 carries out predetermined signal processing on a received image signal, as in the first embodiment. In addition, the timing generator 22 times some operations of the components of the endoscope system 100, as in the first embodiment. In addition, the system controller 23 controls the operations of all components of the endoscope system 100.

The structure and the function of the light-source unit 40 are the same as those of the first embodiment. However, in contrast to the first embodiment, the shutter driver 46 does not receive the output-period detection signal from the system controller 23.

The electronic endoscope 300 comprises the light guide 31, an imaging device 32, a diffuser lens 33, and an object lens 34, as in the first embodiment. In addition, the electronic endoscope 300 comprises a shutter 36, in contrast to the first embodiment.

The shutter 36 is a liquid crystal device, and mounted on the light-receiving surface of the imaging device 32. The shutter 36 can be switched between transmission of and blocking of light approaching the light-receiving surface. The system controller 23 controls the switching operation of the shutter 36.

The imaging device 32 is driven as in the first embodiment, and then, an image signal is generated and transmitted to the image-signal processing circuit 21. The φSL1 and φSLm are transmitted to the system controller 23 via the image-signal processing circuit 21, as in the first embodiment. The system controller 23 orders the shutter 36 to block light since the φSL1 is switched into high state until the φSLm is switched into low state from the high state.

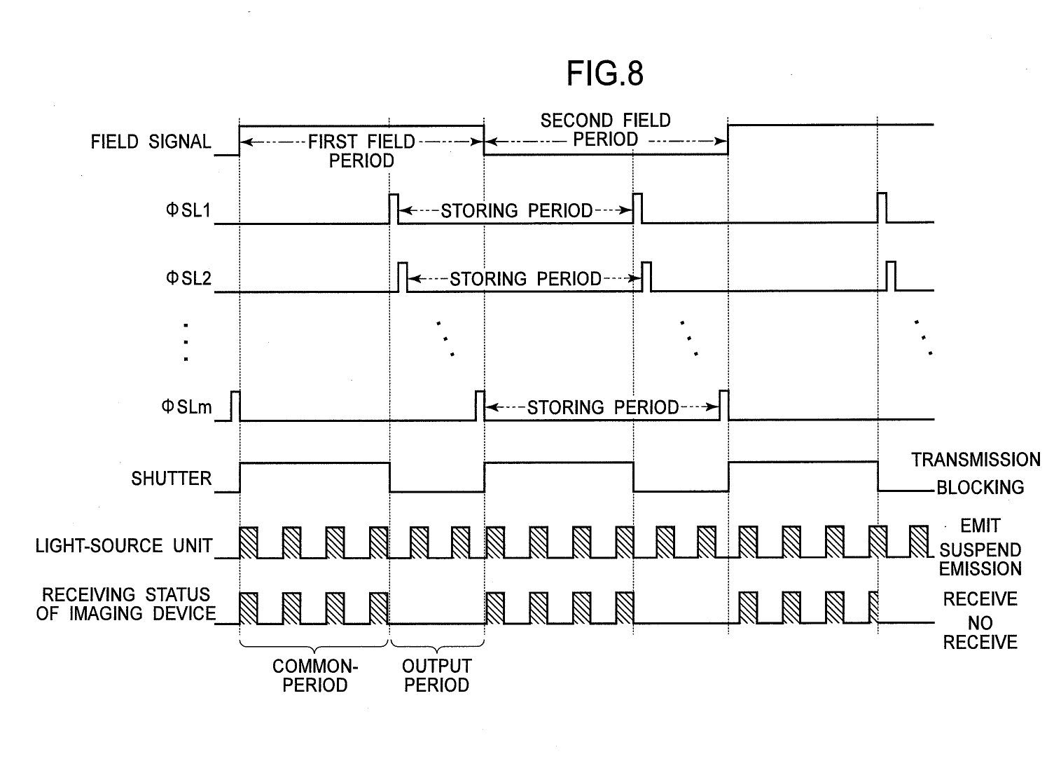

The operation of the imaging device 32 and the shutter 36 in outputting successive fields of image signals is described below with reference to FIG. 8. The operation of the imaging device 32 to output one field of an image signal is same as that of the first embodiment (see FIG. 5).

The field period is divided into a common-period and an output period, as in the first embodiment. The time to switch the field signal between the high and low states is defined as the starting time of the common-period, as in the first embodiment. The period from the completion time of the common period to the time of the next switching of the field signal between the high and low states is defined as the output period, as in the first embodiment.

The combination of a common-period and parts of the output periods before and/or after the common period is defined as a storing period of each row, as in the first embodiment. Signal charges are generated and stored in all the pixels 50 in the corresponding row according to the amount of light received during the storing period for the pixels 50 in the corresponding row. When the φSL of the corresponding row is set to the high state during the output period, the signal charges stored by the PDs 51 are transferred and finally output as pixel signals.

In contrast to the first embodiment, the rotary shutter 42 is driven so that the light-source unit 40 emits a pulse of white light not only during the common-period but also during the output period (see the row of “light-source unit”).

The shutter 36 is ordered to pass an optical image during the common-period. On the other hand, as described above, the shutter 36 is ordered to block the optical image during the output period. Accordingly, an optical image of the reflected light of an object reaches all the pixels 50 in the imaging device 32 only during the common-period, which is the same for all rows during a given field period even though the storing periods differ by row. Consequently, signal charges are actually generated and stored by the pixels in all the rows during the same common-period.

In the second embodiment, when an XY-address type imaging device, such as a CMOS imaging device, is ordered to capture an optical image of a moving object, periods and times during which light is actually received by all the pixels 50 by the line exposure can be made to accord.

In addition, even if an object illuminated by a pulse of white light is photographed and/or filmed without controlling the period when the illumination light is illuminated to the object, uneven luminance for rows caused by the difference of total amounts of illuminated light for rows in a displayed image can be prevented, as in the first embodiment.

A light-source unit which can emit a pulse of light is used in the above first and second embodiments. However, any other light source can be used. In the first embodiment, the same effect can be achieved by suspending emission of light during the output period using a light-source unit which can be switched on and off. For example, a light emission diode can be used as a light source. In the second embodiment, because the shutter 36 is switched between transmission and blockage of an optical image to the light-receiving surface, a light source other than one which can be switched on and off may be adopted.

The light-source unit 40 is mounted in the endoscope processor 20 and 200 in the above first and second embodiments, respectively. However, the light-source unit 40 may be another apparatus separated from the endoscope processor 20 and 200.

The light-source control system in the first embodiment and the shutter control system in the second embodiment are adopted to the endoscope system. The light-source control system and the shutter control system can be adopted to another image capturing apparatus. For example, the same effect can be achieved by adopting the light-source control system to the camera for photographing and/or filming a dark scene with the light-source unit. In addition, the same effect can be achieved even if the shutter control system is adopted to a regular camera.

The pixels 50 are arranged in a grid in the above first and second embodiments. However, the pixels 50 may be arranged in a first and second direction, which are different from each other, as long as a series of pixel signals corresponding to pixels 50 arranged in a given row in the first direction is output in order of the rows in the first direction. In addition, a series of pixel signals corresponding to pixels in a given row is output in row order, in the above first and second embodiments. However, a series of pixel signals corresponding to pixels in a given column may be output in column order.

A CMOS imaging device is used in the above first and second embodiments. However, the same effect can be achieved with any other XY-address type imaging devices.

Although the embodiments of the present invention have been described herein with reference to the accompanying drawings, obviously many modifications and changes may be made by those skilled in this art without departing from the scope of the invention.

The present disclosure relates to object matter contained in Japanese Patent Application No. 2007-314996 (filed on Dec. 5, 2007), which is expressly incorporated herein, by reference, in its entirety.

1. A light-source control system, comprising:
a detector that detects an output period if an XY-address type imaging device is driven to generate an image signal, said XY-address type imaging device comprising a plurality of pixels which are arranged in first and second directions, said image signal comprising a plurality of pixel signals corresponding to said pixels, said pixel signals varying according to signal charges, said pixels generating said signal charges according to the amount of light received during a storing period, said storing period comprising a common-period and a variable-period, said common-period being simultaneous for all of pixel-rows, said variable-period varying according to each of pixel-rows, said pixel-rows comprising said pixels that are arranged in said first direction, series of said pixel signals corresponding to said pixels that are arranged in said same pixel-row being output in order of said pixel-rows during said output period following said common-period; and
a controller that controls a light-source to suspend the emission of illumination light during said output period, said illumination light being shone on an object of which image is captured by said XY-type imaging device.

2. A light-source control system, according to claim 1, wherein said controller orders said light-source to emit a pulse of said illumination light.

3. A light-source control system, according to claim 1, wherein said XY-address type imaging device is mounted in an electronic endoscope.

4. A shutter control system, comprising:
a detector that detects an output period if an XY-address type imaging device is driven to generate an image signal, said XY-address type imaging device comprising a plurality of pixels which are arranged in first and second directions, said image signal comprising a plurality of pixel signals corresponding to said pixels, said pixel signals varying according to signal charges, said pixels generating said signal charges according to the amount of light received during a storing period, said storing period comprising a common-period and a variable-period, said common-period being simultaneous for all of pixel-rows, said variable-period varying according to each of said pixel-rows, said pixel-rows comprising said pixels that are arranged in said first direction, series of said pixel signals corresponding to said pixels that are arranged in said same pixel-row being output in order of said pixel-rows during said output period following said common-period; and
a controller that controls a shutter to block light toward said XY-address type imaging device during said output period, said shutter being mounted on a light-receiving surface of said XY-address type imaging device.

5. A shutter control system, according to claim 4, wherein said XY-address type imaging device is mounted in an electronic endoscope.

6. An endoscope processor, comprising:
a first controller that controls an XY-address type imaging device to generate an image signal, said XY-address type imaging device comprising a plurality of pixels which are arranged in first and second directions, said image signal comprising a plurality of pixel signals corresponding to said pixels, said pixel signals varying according to signal charges, said pixels generating said signal charges according to the amount of light received during a storing period, said storing period comprising a common-period and a variable-period, said common-period being simultaneous for all of pixel-rows, said variable-period varying according to each of said pixel-rows, said pixel-rows comprising said pixels that are arranged in said first direction, series of said pixel signals corresponding to said pixels that are arranged in said same pixel-row being output in order of said pixel-rows during said output period following said common-period; and
a second controller that controls a light-source to suspend the emission of illumination light during said output period, said illumination light being shone on an object of which image is captured by said XY-type imaging device.

7. An endoscope processor, comprising:
a first controller that controls an XY-address type imaging device to generate an image signal, said XY-address type imaging device comprising a plurality of pixels which are arranged in first and second directions, said image signal comprising a plurality of pixel signals corresponding to said pixels, said pixel signals varying according to signal charges, said pixels generating said signal charges according to the amount of light received during a storing period, said storing period comprising a common-period and a variable-period, said common-period being simultaneous for all of pixel-rows, said variable-period varying according to each of said pixel-rows, said pixel-rows comprising said pixels that are arranged in said first direction, series of said pixel signals corresponding to said pixels that are arranged in said same pixel-row being output in order of said pixel-rows during said output period following said common-period; and
a second controller that controls a shutter to block light toward said XY-address type imaging device during said output period, said shutter being mounted on a light-receiving surface of said XY-address type imaging device.

8. An endoscope system, comprising:
an electronic endoscope that comprises an XY-address type imaging device, said XY-address type imaging device comprising a plurality of pixels which are arranged in first and second directions;
a first controller that controls said XY-address type imaging device to generate an image signal, said image signal comprising a plurality of pixel signals corresponding to said pixels, said pixel signals varying according to signal charges, said pixels generating said signal charges according to the amount of light received during a storing period, said storing period comprising a common-period and a variable-period, said common-period being simultaneous for all of pixel-rows, said variable-period varying according to each of said pixel-rows, said pixel-rows comprising said pixels that are arranged in said first direction, series of said pixel signals corresponding to said pixels that are arranged in said same pixel-row being output in order of said pixel-rows during said output period following said common-period;
a light-source that emits illumination light shone on an object of which image is captured by said XY-type imaging device, said light-source being able to be switched between lighting on and off; and
a second controller that controls said light-source to suspend the emission of said illumination light during said output period.

9. An endoscope system, comprising:
an electric endoscope that comprises an XY-address type imaging device, said XY-address type imaging device comprising a plurality of pixels which are arranged in first and second directions;
a first controller that controls said XY-address type imaging device to generate an image signal, said image signals comprising a plurality of pixel signals corresponding to said pixels, said pixel signals varying according to signal charges, said pixels generating said signal charges according to the amount of light received during a storing period, said storing period comprising a common-period and a variable-period, said common-period being simultaneous for all of pixel-rows, said variable-period varying according to each of said pixel-rows, said pixel-rows comprising said pixels that are arranged in said first direction, series of said pixel signals corresponding to said pixels that are arranged in said same pixel-row being output in order of said pixel-rows during said output period following said common-period;
a shutter that is mounted on a light-receiving surface of said XY-address type imaging device, said shutter can be switched between blocking and not blocking light toward said XY-address type imaging device; and
a second controller that controls said shutter to block light toward said XY-address type imaging device during said output period.
